(19) United States
(12) Patent Application Publication
(10) Pub. No.: US 2017/0128216 A1
(43) Pub. Date: May 11, 2017

(54) Prostheses and methods for acetabular rim restoration in quadruped mammals

(71) Applicant: David R. Luck, Denver, CO (US)

(72) Inventor: David R. Luck, Denver, CO (US)

(21) Appl. No.: 14/757,013

(22) Filed: Nov. 9, 2015

(51) Int. Cl. A61F 2/34

(52) U.S. Cl. CPC A61F 2/34; A61F 2002/343; A61F 2310/00023; A61F 2310/00179; A61F 2310/00353; A61F 2310/00359

(57) ABSTRACT

Prostheses and methods for dorsal acetabular rim restoration at the acetabulum of the pelvis of a quadruped mammal, for example for treatment of canine hip dysplasia, are disclosed. The prosthesis includes a unitary implantable structure defining a pelvic portion and a concave portion with an arcuate rim extending from and at parts elevated relative to the pelvic portion. The pelvic portion is adapted for securement at an implant site utilizing suitable means.

### FIELD OF THE INVENTION

This invention relates to treatment of hip joint defects in quadruped mammals and, more particularly, relates to devices and methods for surgical repair/restoration of the dorsal acetabular rim to treat hip dysplasia.

### BACKGROUND OF THE INVENTION

The mammalian hip joint is a main weight-bearing joint connecting the pelvis to the lower extremities and consists principally of a ball and socket. In the normal anatomy, the thigh bone (the femur) is connected to the pelvis at the hip joint (a somewhat imprecise term describing the region of connection of the femur to the pelvis below the ilium and known as the coxofemoral joint). The femoral head is defined at one end of the femur and extends angularly from the femur between the lesser and greater trochanter. The roughly spherical end of the femoral head (the caput, or caput ossis femoris) is the ball which is maintained in the acetabulum, a concave socket-like structure formed during the embryonic process of fusion of the ilium, the ischium, the pubis and the acetabular bone. Cartilage covering the bony surfaces of the femoral head and of the acetabulum ensures a smooth fit and wide range of motion. The articular cartilage covering the femoral head is hard but somewhat sponge-like structurally, with a slick hard surface facing the interior of the joint. In the normal joint, the articular cartilage can change shape slightly when force is applied to it to spread and distribute force more evenly into the subchrondal bone directly beneath the articular cartilage.

Close conformity between the acetabulum and the femoral head throughout their growth period is the ideal for healthy hip joint development and this is how most juvenile hip joints start out (i.e., dysplastic and non-dysplastic hips are mostly indistinguishable in puppies for example). The joint capsule is a fibrous structure filled with synovial fluid that surrounds, isolates, lubricates and protects the joint and is essential to proper development and functioning of the joint. The cushioning effect and fluid pressure of the synovial fluid and the elasticity of the fibrous capsule structure helps to stabilize the joint and keep out contaminants. Moreover, the tissues within the joint extract nutrients from the synovial fluid in which they are bathed.

Most ball and socket joints are formed to allow rotation about three different axes (three degrees of freedom). However, the canine hip joint has a fourth degree of freedom wherein the femoral head may be displaced laterally from the acetabulum. While this is necessary to allow a full range of hind appendage motion, too much laxity in the joint can result joint deterioration in some cases, particularly in patients genetically predisposed to joint diseases such as hip dysplasia or other conditions (osteoarthritis and rheumatoid arthritis, for example) or having undergone some traumatic injury.

Hip dysplasia is a malformation of the hip joint. For example, canine hip dysplasia is described as a deficiency of the dorsal rim of the acetabulum and/or subsequent changes to the size and shape of the femoral head, causing laxity in the hip joint. This condition arises during joint development when the femoral head fails to fit well into the acetabulum. In early phases this can be diagnosed by joint laxity on palpation (the femoral head can be felt moving—up and down and/or in and out of the acetabulum). Treatments for canine hip dysplasia are often not ordered when diagnosed early either due to cost constraints or poor understanding of the long range complications by the owner. Over time, such anatomical abnormalities of the hip joint can result in arthritis and acetabular/femoral head deformity. A common example is deterioration of the dorsal rim of the acetabulum. The joint capsule thickens and becomes inflamed, osteophytes (bone spurs) develop, cartilage erodes, the acetabulum begins to fill in with bone and the femoral head may even luxate (slide out of the acetabulum).

In cases where the femoral head stays somewhat within the acetabulum, the animal may continue without intervention for a long period, arthritic changes continuing over years. It is then usually diagnosed by gait changes, physical palpation, pain with movement, and ultimately by various radiographic techniques. Hip dysplasia remains prevalent in large breed dogs. FIGS. 1 through 3 are provided to give a background view of both healthy (FIG. 1) and unhealthy (dysplastic in this case, FIGS. 2 and 3) hip joint structures.

Current treatment for canine hip dysplasia depends on animal age at time of diagnosis. With very early diagnosis (four weeks) juvenile pubic symphysiodesis can be used. With later diagnosis (between about six to 12 months) triple pelvic osteotomy or DARthroplasty are indicated. Over that age, total hip replacement or femoral head osteotomy are the primary treatment options currently available. Treatment of young juvenile patients is preferred, although cost restraints for total hip replacement and physical restraints for triple pelvic osteotomy interfere with adequate treatment of this age group of animals. DARthroplasty has been successful, but is not a well-known or common procedure in veterinary medicine possibly due to its complexity.

DARthroplasty is a grafting procedure wherein bony graft material from the patient's wing of the ilium is harvested and formed into a cup shape that is then attached over the femoral head at the acetabulum with the expectation that the grafts will form to the deficient dorsal rim of the acetabulum, thus increasing the depth of the dorsal rim and eliminating the laxity of the previously deficient hip joint. In approximately 25% of DARthroplasty patients no bony acetabular addition is formed either due to absorption of the bone graft material or lack of attachment to the deficient acetabular rim.

Femoral head and neck ostectomy and total hip arthroplasty are the major late stage surgical options available to the owner of a dysplastic, arthritic animal. These surgical options for the treatment of canine hip dysplasia have, for the most part, been adaptations of human orthopedic procedures. Femoral head and neck ostectomy is an excision of the femoral head and neck. The procedure has significant potential for long-term complications and often exhibits prolonged recovery, muscle atrophy and skin ulcers in the patient. Best results are achieved and fewer complications are encountered when the animal weighs less than 50 pounds (most effective for dogs less than 35 pounds) thus further complicating the procedure for animals weighing more (often requiring sewing a muscle “sling” or “pad” to support and cushion the femoral shaft using the biceps femoris muscle or part of the gluteal muscle, or using the joint capsule itself to accomplish this).

Total hip arthroplasty uses modular components to optimize the fit of a prosthesis (the BIOMEDTRIX system for example). Femoral neck, shaft and acetabular cup components are selected to fit the patient, the surgical installation typically allowing early if not immediate post-operative use of the limb (a two month recovery period being common to return of normalcy). The procedure is, however, expensive and results, though good, are not guaranteed. Complications due to infection are the most common difficulty experienced with hip replacement (others are known, such as aseptic loosening of implants, related to fibrous membrane development between the bone and the cement used in the surgery, and sciatic neuropraxia (the inability of the nerve to conduct impulses)).

Other treatment alternatives for a variety of diseased or damaged joints in mammals have also heretofore been suggested and/or utilized, including surgical installation of various types of implants and/or prostheses (see, for example, U.S. Pat. Nos. 7,799,077 and 5,047,056 and U.S. Patent Application Publication Nos. 2013/0226252 and 2013/0190886). These however have not been shown to be readily adaptable to problems associated with treatment of hip dysplasia or related disabilities due to malformation of the acetabular rim of the acetabulum structure. It is therefore apparent from the foregoing that further improvements could still be utilized in this field, particular directed to stable and long lasting outcomes in repair of acetabular rim deformities in quadruped mammal hip joints that minimize surgical impact on the animal.

### SUMMARY OF THE INVENTION

This invention provides prostheses and methods for dorsal acetabular rim restoration at the acetabulum of the pelvis of a quadruped mammal, for example for treatment of canine hip dysplasia. The prostheses and methods are inexpensive relative to many other procedures and provide safe and effective treatment mechanisms resulting in stable and long lasting outcomes while minimizing recovery times and complications.

A prosthesis in accord with this invention includes a pelvic portion with a surface adapted for securement at the pelvis of the mammal adjacent to the acetabulum at implant site securement structure. A concave portion (defining a concavity) of the prosthesis includes an arcuate rim extends from and is elevated at parts relative to the pelvic portion. Securement means is provided for securing the prosthesis at the implant site.

The methods of this invention are adapted for deficient dorsal acetabular rim restoration in a bony pelvis of a quadruped mammal. The method includes steps for measuring the deficient dorsal acetabular rim to provide a profile and surgically exposing the deficient dorsal acetabular rim. The deficient dorsal acetabular rim is prepared to establish an implant site and a suitably configured artificial acetabular rim restoration prosthesis is selected from the profile. The suitably configured artificial acetabular rim restoration prosthesis is then secured at the implant site.

By these means, surgically implantable prostheses and methods are provided that would require no harvesting of the boney material from the ilium, that when attached to the deficient dorsal acetabular rim would establish a positively fixed implant giving added depth to the acetabulum providing near normal support for the hip joint, that would lessen the complexity of the surgical procedures and recovery times, and that would reduce morbidity currently seen in other treatments. The implantable prosthesis can, due to its simplicity, be made in a large variety of sizes to conform to differences in diagnosed cases. The pelvic portion may be configured with micro-pores to allow bony ingrowth from a properly surgically prepared dorsal acetabular rim.

It is therefore an object of this invention to provide prostheses and methods for dorsal acetabular rim restoration at the acetabulum of the pelvis of a quadruped mammal.

It is another object of this invention to provide prostheses and methods for dorsal acetabular rim restoration for treatment of canine hip dysplasia.

It is still another object of this invention to provide prostheses and methods for dorsal acetabular rim restoration that are inexpensive, safe and effective.

It is yet another object of this invention to provide prostheses and methods for dorsal acetabular rim restoration that enables stable and long lasting treatment outcomes while minimizing recovery times and complications from surgery.

It is still another object of this invention to provide prostheses and methods for dorsal acetabular rim restoration that require little or no bony material harvesting while establishing a positively fixed restoration to add depth to the acetabulum providing near normal support for the hip joint, that would lessen the complexity of the surgical procedures and recovery time, and that would reduce morbidity currently seen in other treatments.

It is yet another object of this invention to provide that would require no harvesting of the boney material from the animal, that establishes added depth to the acetabulum, and that provides near normal support for the hip joint.

It is still another object of this invention to provide prostheses and methods for dorsal acetabular rim restoration that lessens complexity of surgical procedures and recovery times involved and reduces morbidity.

It is another object of this invention to provide a prosthesis for dorsal acetabular rim restoration at the acetabulum of the pelvis of a quadruped mammal that includes a pelvic portion with a surface adapted for securement at the pelvis adjacent to the acetabulum and a concave portion with an arcuate rim extending from and elevated at parts relative to the pelvic portion.

It is still another object of this invention to provide a prosthesis for treatment of canine hip dysplasia that includes a first portion with a pelvic implant site securement structure, a second portion defining a concavity having an arcuate rim adaptation extending from and elevated at parts relative to the first portion, and securement means for securing the prosthesis at the securement structure of the first portion at an implant site.

It is yet another object of this invention to provide a method for deficient dorsal acetabular rim restoration in a bony pelvis of a quadruped mammal that includes the steps of measuring the deficient dorsal acetabular rim to provide a profile, surgically exposing the deficient dorsal acetabular rim, preparing the deficient dorsal acetabular rim to establish an implant site, selecting a suitably configured artificial acetabular rim restoration prosthesis from the profile, and securing the suitably configured artificial acetabular rim restoration prosthesis at the implant site.

With these and other objects in view, which will become apparent to one skilled in the art as the description proceeds, this invention resides in the novel construction, combination, and arrangement of parts and methods substantially as hereinafter described, and more particularly defined by the appended claims, it being understood that changes in the precise embodiment of the herein disclosed invention are meant to be included as come within the scope of the claims.

### BRIEF DESCRIPTION OF THE DRAWINGS

The accompanying drawings illustrate a complete embodiment of the invention according to the best mode so far devised for the practical application of the principles thereof, and in which:

FIG. 1 is a perspective view of a portion of a healthy canine pelvic bone;

FIG. 2 is a perspective view of a pelvic bone portion as shown in FIG. 1 but exhibiting deterioration of the acetabular rim typical of hip dysplasia;

FIG. 3 is a perspective view of a canine hip joint with a deteriorated acetabulum as shown in FIG. 2;

FIG. 4 is a bottom perspective view of the prosthesis of this invention;

FIG. 5 is a side perspective view of the prosthesis of this invention;

FIG. 6 is another side perspective view of the prosthesis of this invention;

FIG. 7 is a top perspective view of the prosthesis of this invention;

FIG. 8 is a perspective view of the prosthesis of this invention being implanted at the acetabular rim of the pelvic bone portion shown in FIG. 2;

FIG. 9A is a perspective view of portions of the canine hip joint of FIG. 3;

FIG. 9B is a sectional view taken through section lines B-B of FIG. 9A;

FIGS. 9C and 9D are perspective views of the left and right sides, respectively, of a pelvic bone/hip joint each having an implanted prosthesis of this invention;

FIG. 9E is a sectional view taken through section lines E-E of FIG. 9D; and

FIGS. 10 and 11 are perspective view illustrations of implanting of the prosthesis of this invention.

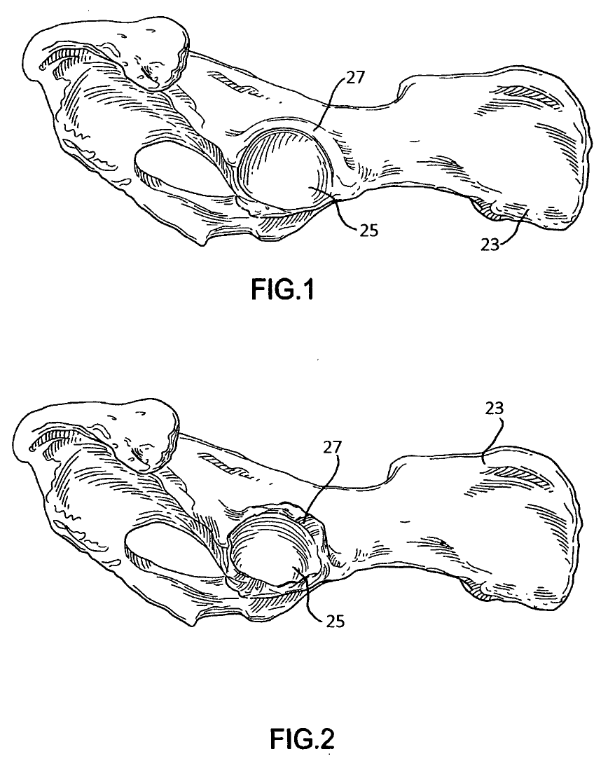

### DESCRIPTION OF THE INVENTION

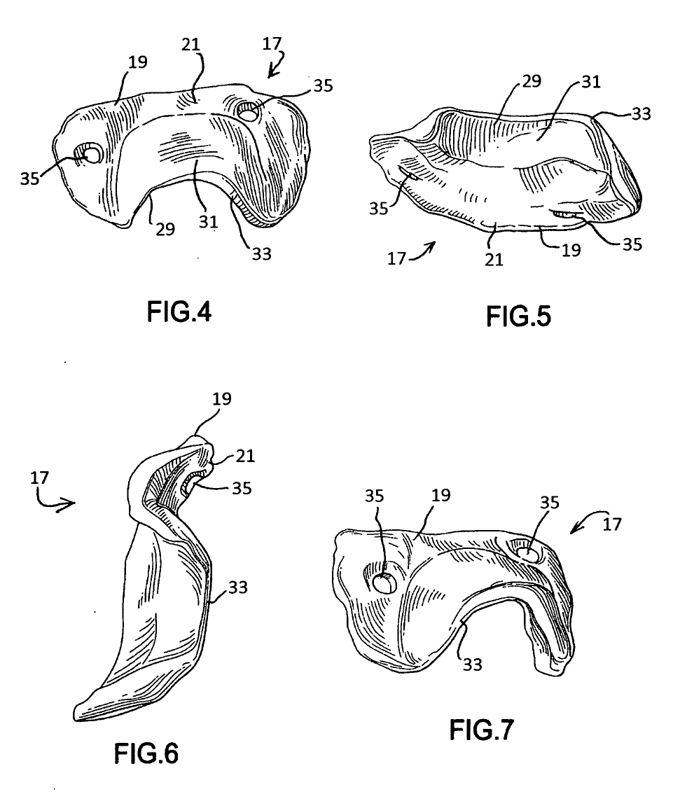

FIGS. 4 through 7 show a currently preferred embodiment of prosthesis 17 of this invention. Initially conceived of to treat symptoms of hip dysplasia in canines, the prostheses and methods described herein can be adapted for use in any quadruped mammal that develops deficiency of the dorsal acetabular rim similar to the deficiency produced by canine hip dysplasia (for example, by inheritance, disease or trauma). Thus, while the description herein utilizes canine examples and skeletal/physiological terminology, it should be understood to apply to any such deficiency in other quadruped mammals.

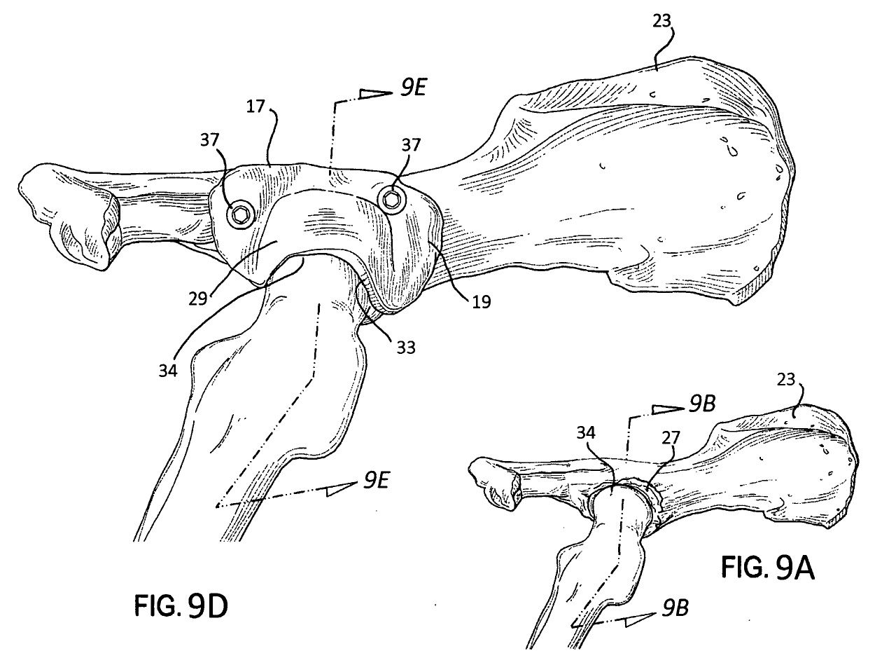

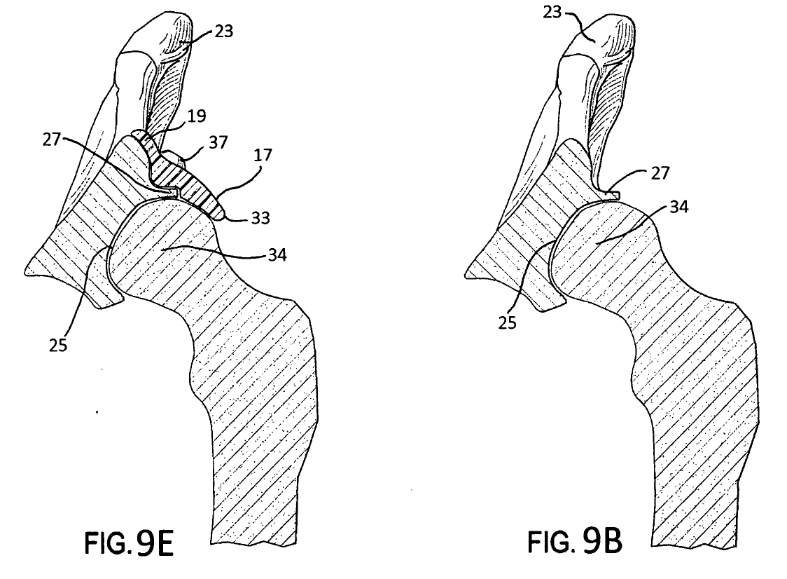

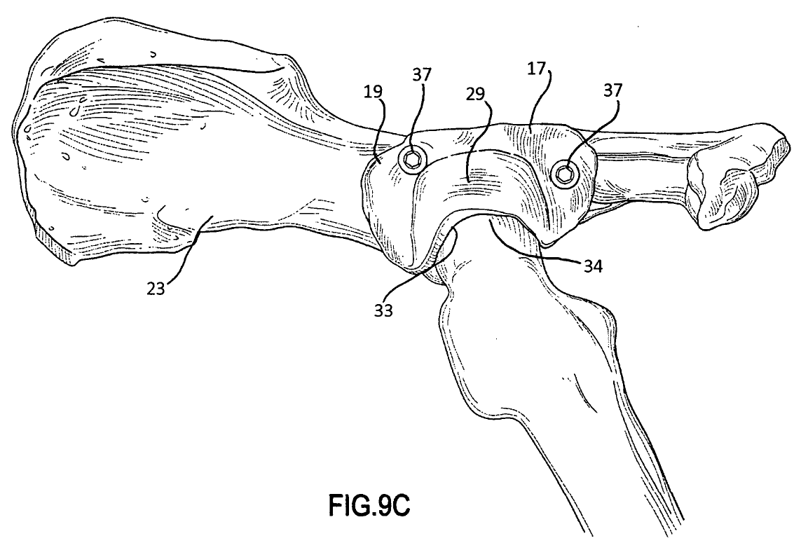

Prosthesis 17 includes a unitary implantable structure adapted for dorsal acetabular rim restoration at the acetabulum of the pelvis of the animal presented for treatment. Pelvic portion 19 has a bottom surface 21 that is adapted (both surface contour and size) for securement at pelvic bone 23 adjacent (dorsal) to the acetabulum 25 and dorsal rim 27. Concave portion 29 defines concavity 31 and arcuate rim 33 (a dorsal acetabular rim adaptation) and is adapted and configured for femoral head 34 engagement as shown in FIGS. 9C, 9D and 9E. Portion 29 (and thus concavity 31 and rim 33) extends from pelvic portion 19 and is elevated at parts relative to portion 19 (and thus pelvic bone 23 once implanted). As used herein the term “elevated” is not to be understood to mean at a higher altitude in any geographically absolute sense, but only to mean shifted positionally relative to surface 21 so as to be offset from pelvic bone 23 once implanted.

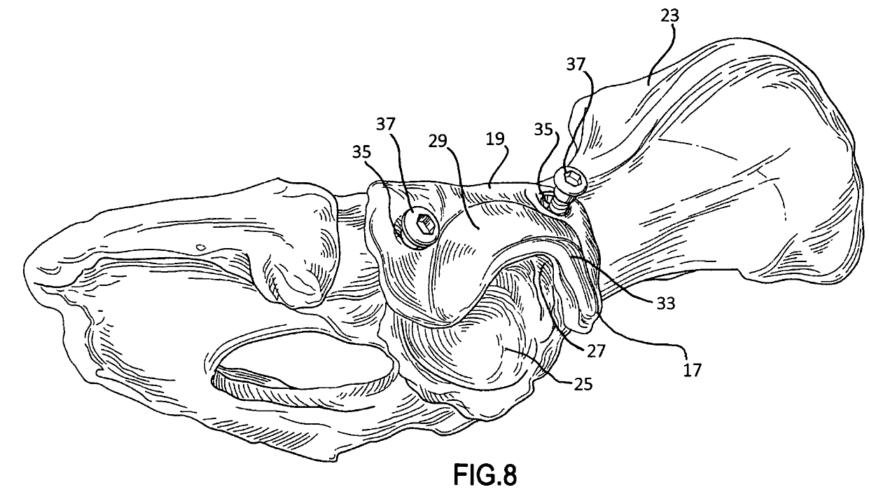

In this embodiment of prosthesis 17, pelvic portion 19 includes openings 35 for bone screws for securement of the prosthesis at the implant site. This securement structure feature could however be replaced (for example, by structure or surface preparation at surface 21 adapted to achieve long lasting securement using known bone cements). Suitable means for securement of prosthesis 17 at the implant site are provided (for example, bone screws 37 as shown in FIGS. 8 and 9) adapted to assure long term immobility of the prosthesis relative to pelvic bone 23.

The prostheses 17 can be made of any material not likely to be rejected including plastics, metals, ceramics, biological materials (such as collagen or other extracellular matrix materials), hydroxyapatite, cellular materials (such as stem cells, chondrocyte cells or the like) or combinations thereof. At present, the preferred material is ultra-high-molecular-weight polyethylene. This material may be used alone in formation of prostheses 17, or could be established on a titanium mesh or foam framework. Whatever material is used, it should be semi-rigid while exhibiting a selected extent of flexibility (particularly at pelvic portion 19) to conform to pelvic implantation securement site variations which may be encountered. That is, the material used should provide the hardness needed to maintain contact with femoral head 34 while yet resisting excessive wear to either the prosthesis or the femoral head. The material selected may also usefully be selected to provide micro-porous structure, at least at surface 21 thereof, adapted to allow bony ingrowth from a properly surgically prepared dorsal acetabular rim 27 to provide a more durable mending and long term strength between prosthesis 17 and bone 23 at the surgical implant site. A variety of sizes can be provided to conform to differences in diagnosed cases that may be encountered. Prostheses 17 can be configured for either left or right side acetabular rim/hip joint repair (as shown in FIGS. 9C and 9D, respectively).

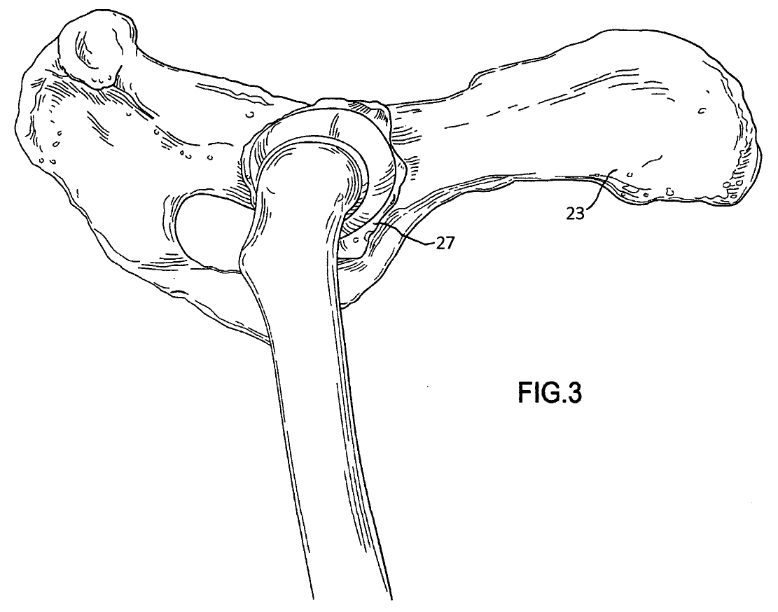

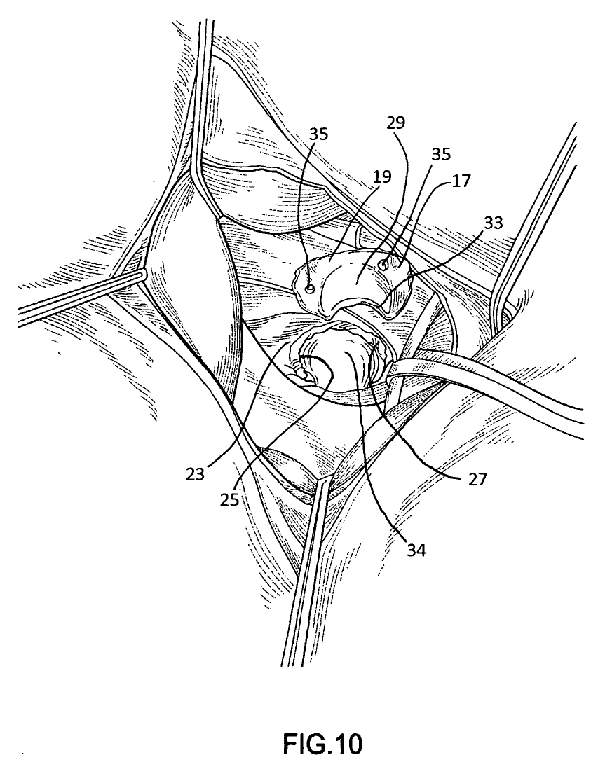

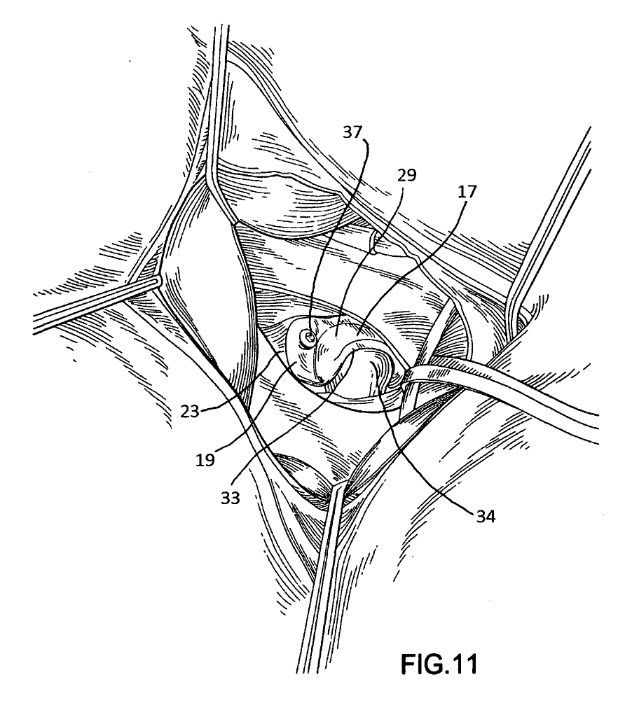

Turning now to FIGS. 9 through 11, a description of methods adapted to surgical implant of the prostheses of this invention will proceed. The deficient dorsal acetabular rim 27 (see FIGS. 2, 3, 8 and 9A) at the patient's bony pelvis is appropriately measured providing an implant site profile. These measurements and profile help in selection of implant size and, in some cases, surface 21 site orientation. Non-invasive measurement, for example utilizing well known radiographic techniques providing extended and DAR views, is preferred though in-situ measurement during the procedure may be employed instead or in addition. CT imaging, MRI, or ultrasound could instead be used. The deficient dorsal acetabular rim may be surgically exposed and the implant site properly prepared once a suitably configured artificial acetabular rim restoration prosthesis has been selected fitting the profile. The prosthesis is located at the bony pelvis implant site through a surgical incision using, for example, posterior, muscle splitting techniques. Alternatively, prosthesis location and securement techniques could be developed utilizing endoscopic methods and instrumentation. Once located, the prosthesis is secured at the pelvic bone as heretofore described, for example by forming screw receiving holes in the bony pelvis at the implant site corresponding to the securement openings in and desired orientation of the prosthesis. Once secured, the site is cleaned and prepared for closing, and the various required incisions are then closed.

As may be appreciated from the foregoing, this invention provides prostheses and methods for dorsal acetabular rim restoration whereby stable and long lasting rim/joint repair is achieved with less discomfort and risk for the animal. This positively fixed restoration adds depth to the acetabulum thereby providing near normal support for the hip joint.

What is claimed is:

1. A prosthesis for dorsal acetabular rim restoration at the acetabulum of the pelvis of a quadruped mammal comprising a pelvic portion with a surface adapted for securement at the pelvis adjacent to the acetabulum and a concave portion including an arcuate rim extending from and elevated at parts relative to said pelvic portion.

2. The prosthesis of claim 1 made of at least one of plastic, metal, ceramic, biological extracellular matrix material, hydroxyapatite, and cellular material.

3. The prosthesis of claim 1 made of ultra-high-molecular-weight polyethylene.

4. The prosthesis of claim 3 wherein the ultra-high-molecular-weight polyethylene is established on a titanium mesh or foam framework.

5. The prosthesis of claim 1 made of semi-rigid material having a selected extent of flexibility to conform to pelvic implantation securement site variations.

6. The prosthesis of claim 1 wherein said pelvic portion includes openings, the prosthesis further comprising bone screws adapted for receipt through said openings.

7. The prosthesis of claim 1 wherein said pelvic portion includes micro-porous structure at least at said surface thereof adapted to allow boney ingrowth.

8. A prosthesis for treatment of canine hip dysplasia comprising:
a first portion including a pelvic implant site securement structure;
a second portion defining a concavity with an arcuate rim adaptation and extending from said first portion; and
securement means for securing the prosthesis at said securement structure of said first portion at an implant site.

9. The prosthesis of claim 8 manufactured in various sizes to conform to a variety of differences in diagnosed cases of canine hip dysplasia,

10. The prosthesis of claim 8 wherein said securement means includes at least one of bone screws and bone cement.

11. The prosthesis of claim 8 wherein said first portion and said second portion are a unitary structure.

12. The prosthesis of claim 8 wherein said first portion exhibits a selected extent of flexibility and wherein said securement structure and said securement means are adapted for immobility of the prosthesis.

13. The prosthesis of claim 8 wherein said arcuate rim adaptation is a dorsal acetabular rim adaptation, said concavity and said rim adaptation configured for femoral head engagement.

14. A method for deficient dorsal acetabular rim restoration in a bony pelvis of a quadruped mammal comprising the steps of:
measuring the deficient dorsal acetabular rim to provide a profile;
surgically exposing the deficient dorsal acetabular rim;
preparing the deficient dorsal acetabular rim to establish an implant site;
selecting a suitably configured artificial acetabular rim restoration prosthesis from the profile; and
securing the suitably configured artificial acetabular rim restoration prosthesis at the implant site.

15. The method of claim 14 wherein the step of measuring the deficient dorsal acetabular rim is performed non-invasively.

16. The method of claim 14 wherein the step of securing an artificial acetabular rim restoration prosthesis includes securing a pelvic portion of the prosthesis at the implant site, the prosthesis being semi-rigid with the at least the pelvic portion exhibiting a selected extent of flexibility.

17. The method of claim 14 wherein the step of securing an artificial acetabular rim restoration prosthesis includes forming screw receiving holes in the bony pelvis of the mammal at the implant site corresponding to securement openings in the prosthesis.

18. The method of claim 14 wherein the step of selecting a suitably configured artificial acetabular rim restoration prosthesis includes configuring the prosthesis with a pelvic portion having micro-porous structure adapted to allow boney ingrowth.

19. The method of claim 14 wherein the step of surgically exposing the deficient dorsal acetabular rim includes one of making a surgical incision using a posterior, muscle splitting technique or exposing the deficient rim endoscopically.

20. The method of claim 14 wherein the step of selecting a suitably configured artificial acetabular rim restoration prosthesis includes configuring the prosthesis using at least one of plastic, metal, ceramic, biological extracellular matrix material, hydroxyapatite, and cellular material.
